(19) United States
(12) Patent Application Publication
(10) Pub. No.: US 2025/0117722 A1
(43) Pub. Date: Apr. 10, 2025

(54) SOFTWARE AS A SERVICE (SAAS) SYSTEM FOR THE DETERMINATION OF SPECIFIC CALCULATED INDIVIDUAL FUNCTIONAL CHANGES IN THE LEVEL OF RESOURCE NEEDS USING UNIQUE IDENTIFIERS WITHIN ACTIVITIES OF DAILY LIVING, COMMUNITY ENGAGEMENT, SOCIAL, AND EDUCATIONAL ENVIRONMENTS

(71) Applicants: Samuel Bradbury, Santa Fe, NM (US); Sanja Zeman, Pottsville (AU)

(72) Inventors: Samuel Bradbury, Santa Fe, NM (US); Sanja Zeman, Pottsville (AU)

(21) Appl. No.: 18/969,674

(22) Filed: Dec. 5, 2024

Related U.S. Application Data
(60) Provisional application No. 63/588,103, filed on Oct. 5, 2023.

(51) Int. Cl. G06Q 10/0631; H04L 67/10

(52) U.S. Cl. CPC G06Q 10/0631; H04L 67/10

(57) ABSTRACT

The software as a service (SAAS) platform of the present invention analyzes an individual's functional capabilities by comparing self-reported data with professional observations. It utilizes research-based normative data to calculate changes in function across various domains, such as activities of daily living and cognitive function. The system generates reports with graphical representations and detailed analyses of deficits or gains. It also projects long-term status changes and calculates resource requirements, using databases of costs and aids to provide cost analyses. This platform aids in various areas, including disability claims, insurance claims, legal disputes, and rehabilitation planning, by tracking functional changes to optimize resource use and improve outcomes.

### CROSS-REFERENCE TO RELATED APPLICATIONS

The present application claims priority to application No. 63/588,103 filed on Oct. 5, 2023 the disclosure of which is hereby incorporated in its entirety at least by reference.

### BACKGROUND OF THE INVENTION

### 1. Field of the Invention

The present invention generally relates to systems and methods for assessing and analyzing functional capabilities in individuals, but more specifically a software as a service (SaaS) system for the determination of specific calculated individual functional changes in level of resource need using unique identifiers within activities of daily living, community engagement, social, and educational environments.

### 2. Description of Related Art

Accurate assessment of an individual's functional capabilities is critical in various fields, including healthcare, rehabilitation, insurance, and legal sectors. Traditional methods often rely on subjective evaluations or standardized tests that may not capture the full spectrum of an individual's abilities or changes over time. These methods may lack integration of self-reported data with professional observations and often do not utilize normative datasets for comprehensive analysis.

Existing systems may not effectively calculate percentage losses or gains in function across specific domains such as activities of daily living, cognitive function, community engagement, or educational changes. Further, they may not project long-term changes in status or resource requirements, nor provide detailed cost analyses based on current fee schedules.

Consequently, a solution is provided.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present invention is to provide a system configured to integrate multiple data sources, utilizes normative datasets, and provide detailed analyses and visual representations of functional changes over time to create a specific resource requirement worksheet.

It is another object of the present invention to provide a system configured to enhance decision-making processes in rehabilitation planning, resource allocation, claims management, and legal disputes.

In order to do so, a software-as-a-service (SaaS) system for determining specific calculated individual functional changes in the level of resources needed using unique identifiers within activities of daily living, community engagement, social participation, and educational environments are provided, the system comprising an input interface configured to receive data collected from an individual user regarding previous capabilities identified by unique identifiers within specific functional domains including activities of daily living, community engagement, social participation, and educational activities; wherein the input interface is configured to receive observed capability data provided by a trained professional corresponding to the unique identifiers; a first database configured to store normative data comprising research-based datasets of functional levels corresponding to the unique identifiers within the specific functional domains; a second database configured to store fee schedules, costs, aids, home modifications, resource time requirements of normal populations, and diagnosis-specific physical change expectations; a processor configured to: associate the received individual data and professional data with the unique identifiers to map functional capabilities accurately; compare the individual data and the observed capability data to the normative data for each unique identifier to determine deviations from normative functional levels; calculate specific percentage losses or gains in function for the individual user for each unique identifier based on the comparison; determine the level of resource need by integrating the calculated functional changes with time components, resource requirements, and equipment needs corresponding to each unique identifier; generate an outcome report that includes the calculated percentage losses or gains, graphical representations of changes in function, and recommendations for resources, equipment, services, and treatments; project long-term changes in status, function, and resource requirements based on diagnosis-specific expectations; and an output interface configured to output the outcome report to authorized users.

In one embodiment, the unique identifiers comprise specific tasks or activities that uniquely identify functional capabilities and corresponding resource needs within the functional domains. In one embodiment, the input interface presents questionnaires to the individual user formatted with prompts including, “Could you do the following?,” “Did you do the following?,” and “Were you responsible for the following?” to assess pre-injury or baseline capabilities accurately. In one embodiment, the processor combines the data collected from the individual user and the observed capability data provided by the trained professional to assess changes in functional levels more accurately. In another embodiment, the processor is configured to use the data collected from the individual user and the observed capability data and apply a scoring process to objectively assess and score the current status of the individual, integrate the score with the normative data and diagnosis data to perform priority data calculations for determining resource needs.

In one embodiment, the outcome report includes graphical representations to visually explain changes in function based on specific domains and subdomains. In another embodiment, the processor calculates required resources, services, equipment, and associated costs by integrating pre- and post-function time requirements and accessing current fee schedules from the database. In one embodiment, the projection of long-term changes utilizes diagnosis-specific normative data to anticipate future functional changes and resource requirements. In one embodiment, the system is utilized for individual baseline testing, periodic reassessments, evaluations by government agencies to determine functional changes over time, and determination of resource requirements and disability payouts in medical legal cases.

In another aspect of the invention, a method for determining specific calculated individual functional changes in the level of resource need using unique identifiers within activities of daily living, community engagement, social, and educational environments is provided, the method comprising receiving, via an input interface of a software-as-a-service (SaaS) system, data collected from an individual user regarding previous capabilities identified by unique identifiers within specific functional domains including activities of daily living, community engagement, social participation, and educational activities; receiving, via the input interface, observed capability data corresponding to the unique identifiers, the capability data provided by a trained professional; associating the received individual user data and the professional data with the unique identifiers to accurately map functional capabilities; accessing normative data from a first database, the normative data comprising research-based datasets of functional levels corresponding to the unique identifiers within the specific functional domains; accessing additional data from a second database, the additional data comprising fee schedules, costs, aids, home modifications, resource time requirements of normal populations, and diagnosis-specific physical change expectations; comparing the individual user data and the observed capability data to the normative data for each unique identifier to determine deviations from normative functional levels; calculating specific percentage losses or gains in function for the individual user for each unique identifier based on the comparison; determining the level of resource need by integrating the calculated functional changes with time components, resource requirements, and equipment needs corresponding to each unique identifier; generating an outcome report, and outputting the outcome report via an output interface.

The foregoing has outlined rather broadly the more pertinent and important features of the present disclosure so that the detailed description of the invention that follows may be better understood and so that the present contribution to the art can be more fully appreciated. Additional features of the invention, which will be described hereinafter, form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE DRAWINGS

Other features and advantages of the present invention will become apparent when the following detailed description is read in conjunction with the accompanying drawings, in which:

FIG. 1 is a network Internet computer system diagram illustrating the system architecture of the present invention.

FIGS. 2A-D illustrate a flow diagram of the system according to an embodiment of the present invention.

FIGS. 3A-C illustrates an exemplary pre-screening input evaluation according to an embodiment of the present invention.

FIGS. 4A-B illustrate an exemplary screen shot of a user interface for establishing report content with sections and break points for the selected content according to an embodiment of the present invention.

FIGS. 5A-D show exemplary visual display summary graphs for domain category scores according to an embodiment of the present invention.

FIGS. 6A-B show domain sections and summary scores according to an embodiment of the present invention.

FIG. 7 illustrates a sample table of reviewed sub-domains according to an embodiment of the present invasion.

FIG. 8 illustrates a sample table of cost analysis for services, resources, aids, and equipment calculations according to an embodiment of the present invention.

FIG. 9 illustrates a sample aides and home modification table according to an embodiment of the present invention.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide a software as a service (SaaS) system for the determination of specific calculated individual functional changes in level of resource need using unique identifiers within activities of daily living, community engagement, social, and educational environments.

It is to be understood that the terminology used herein is for the purpose of describing particular embodiments only and is not intended to be limiting. The terms “a” or “an,” as used herein, are defined as to mean “at least one.” The term “plurality,” as used herein, is defined as two or more. The term “another,” as used herein, is defined as at least a second or more. The terms “including” and/or “having” as used herein, are defined as comprising (i.e., open language). The term “providing” is defined herein in its broadest sense, e.g., bringing/coming into physical existence, making available, and/or supplying to someone or something, in whole or in multiple parts at once or over a period of time. These terms generally refer to a range of numbers that one of skill in the art would consider equivalent to the recited values (i.e., having the same function or result). In many instances these terms may include numbers that are rounded to the nearest significant figure. A “unique identifier” refers to any specific task, activity, or marker that distinctly and reliably indicates the functional capabilities or resource needs within specified functional domains, enabling precise and personalized assessment and planning.

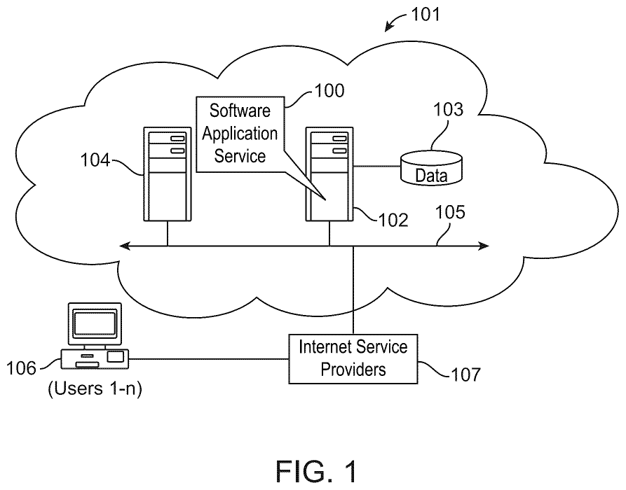

FIG. 1 illustrates the network architecture of the software-as-a-service (Saas) system 100 according to an embodiment of the present invention. The system is connected to the Internet 101 and comprises one or more Internet-connected servers 102 executing the software application from non-transitory media. These servers are linked to a data repository 103, which can be any suitable form of data storage known in the art. The system also includes third-party Internet-connected servers 104 connected to the Internet backbone 105. Although only one third-party server 104 is depicted for simplicity, it is understood that potentially millions of similar servers are connected to the Internet via the Internet backbone 105. Multiple users, represented as users 1 through n (106), connect to the Internet-connected server 102 via an Internet Service Provider (ISP) 107, allowing them to access the system.

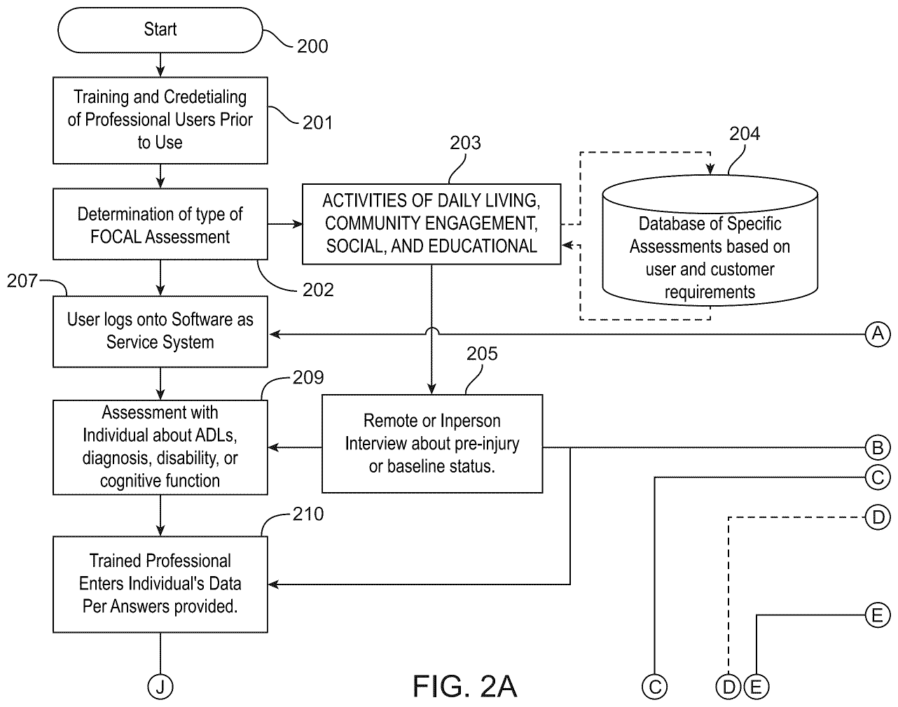

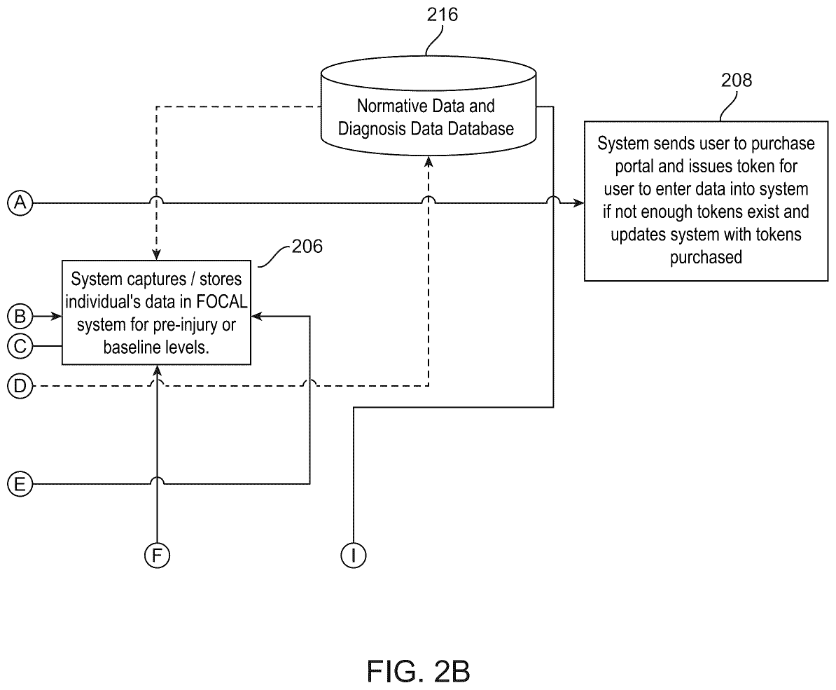

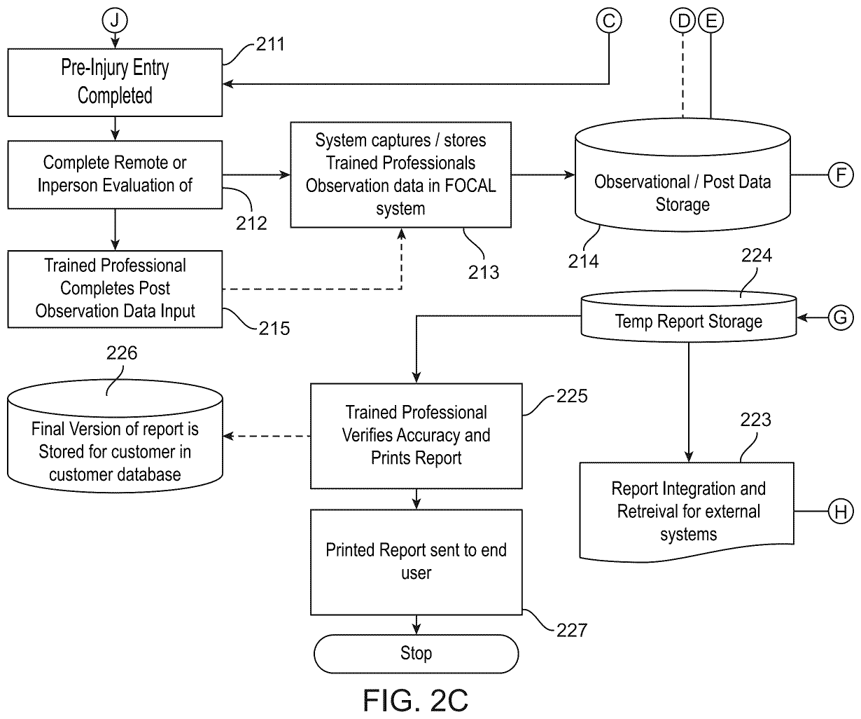

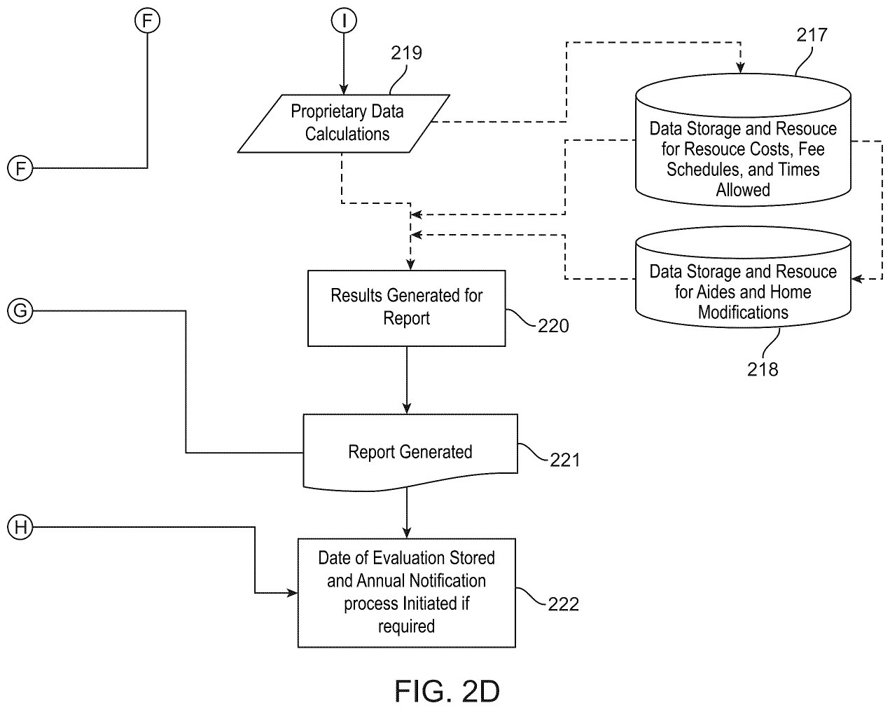

FIGS. 2A-D depict a flow diagram of a method 200 for using the system. The process begins in step 201, where professional users undergo training and credentialing prior to using the system. The system relies on professionals, such as therapists, to perform assessments based on user self-review submissions, which will be elaborated upon later. Ensuring that professional users are verified and trained on system operations is critical to maintain the accuracy of assessment results.

In step 202, a determination is made regarding the type of focal assignment needed for activities of daily living (ADLs), community engagement, social, and educational assessments, as indicated in 203. This step ensures that the end user provides essential data associated with the outcomes of the assessment process. The system offers the capability to evaluate various aspects, including activities of daily living, community engagement, social and cognitive capabilities, and educational expectations or requirements. These evaluations are conducted through the assessment of personal care activities, mobility, instrumental activities, participation, cognitive function, and age- and grade-specific educational evaluations.

In one embodiment, there are four primary areas of specific function: personal care, mobility, instrumental activities, and participation. In step 204, based on the determination made, the system retrieves from a database specific assessments tailored to user and customer requirements. The results of this determination provide a measured level of deficit from previous conditions, normative conditions, or disability-based normative data. This allows the end user to quickly identify areas where resources are needed, the types of resources required, and the associated costs.

Still referring to FIGS. 2A-D, in step 207, a user logs onto the system for an assessment related to ADLs, diagnosis, disability, or cognitive function, as indicated in 209. In some embodiments, a remote or in-person interview is conducted to gather pre-injury or baseline status from the user in step 205, which is then factored into the assessment in step 209. The system captures and stores the individual's data in the FOCAL system for pre-injury or baseline levels in step 205, storing it in the database referred to in step 204.

In step 210, a trained professional enters the user's data based on the answers provided in step 205. If available, pre-injury or baseline levels are entered in step 211 via data collected in step 206. In step 212, a complete remote or in-person evaluation is performed, with the input data stored via step 213. The trained professional completes post-observation data input in step 215, which is also stored via step 213.

Utilizing the inputted evaluation data from the user, the trained professional's post-observation data, pre-injury or baseline data, normative data, and diagnosis data from database 216, the system performs priority data calculations via a system algorithm in step 219. After these calculations are completed, resources related to resource costs, fee schedules, and allowed times are saved in database 217. Additionally, resources for aids and home modifications are saved in database 218.

In step 220, the results are generated, which may be formulated into a report in step 221. This report can be integrated with or made available for retrieval by external systems in step 223. In some embodiments, there is a temporary storage database 224 for the generated report. After the report is generated, the date of evaluation is stored, and the annual notification process is initiated if required, as shown in step 222. The annual notification process provides evaluator entities with notifications when a user is due for an annual re-assessment, which is critical for maintaining benefits and resources. It also allows larger agencies to staff appropriate resources for annual re-assessments. In some embodiments, this entails sending an email notification to the end user.

In step 225, the trained professional verifies the accuracy of the report and prints it, after which the printed report is sent to the end user in step 227. The final version of the report is also stored for the customer in a customer database 226.

In some embodiments, the system performs several data calculations as part of its functionality. It calculates percentage losses or gains in function specific to activities of daily living, community engagement, educational changes, and cognitive function. The system utilizes normative data as a component measure for determining changes in functional levels, combining both individual and trained professional data to assess these changes accurately. To graphically explain changes in function based on specific domains and subdomains, the system employs radar graphs. Algorithms are used to determine levels of functional change and assign ratings to these changes. Time is incorporated as a component to determine required resources, equipment, or treatments, using comparative normal time versus tested time to identify specific resource requirements and client needs. The system performs calculations of deficits or gains by domain and subdomain, integrating pre- and post-time functions to calculate necessary resources, services, and equipment. It also calculates the cost of services for specific periods based on these findings. To ensure accurate and current cost analyses, the system includes databases of fee schedules, costs, aids, home modifications, and resource time requirements of normal populations. Additionally, it maintains a database of specific diagnoses and the expected physical changes associated with them. The system allocates resources based on the individual's true previous function and performed tasks. By projecting long-term changes in status, function, and resource requirements, the system can be used for individual baseline testing, annual or regular time-frame testing, by government agencies to determine functional changes over time, and in medical legal cases to ascertain resource requirements and disability payouts.

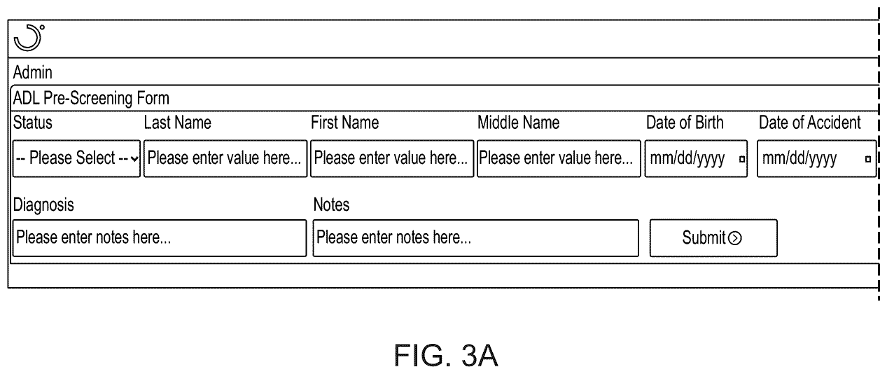

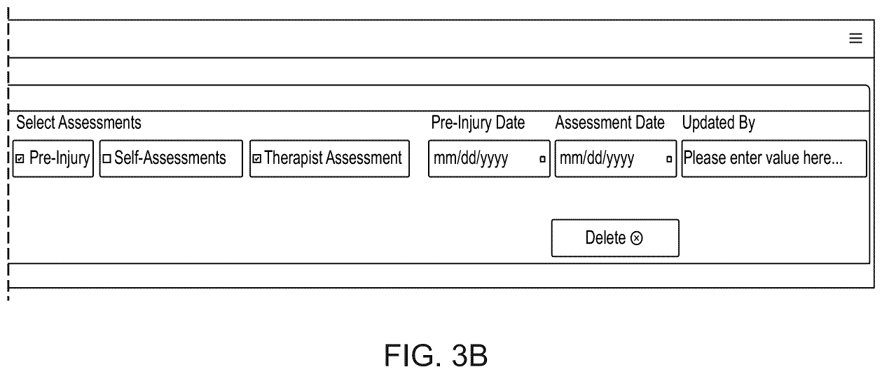

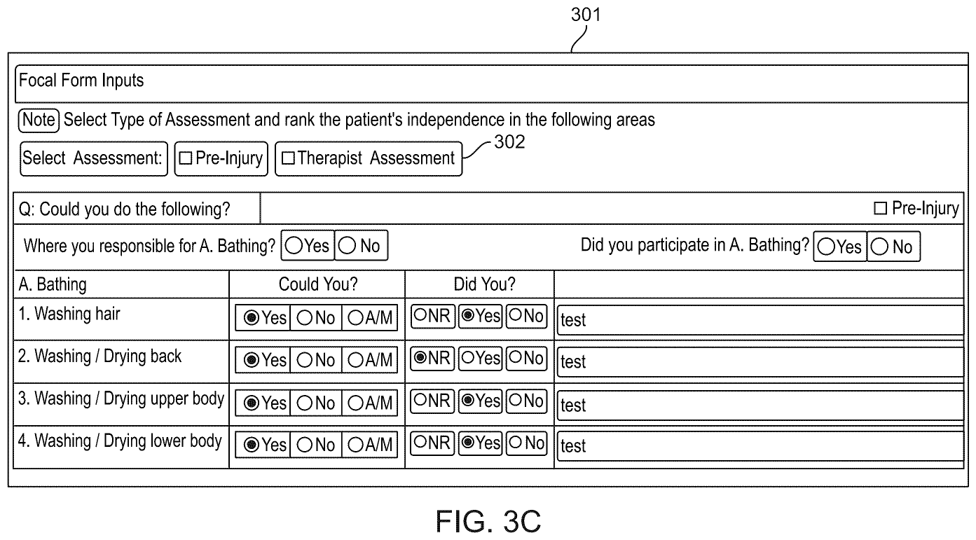

FIGS. 3A-C illustrate pre-screening input evaluation forms displayed after a user logs into the system. The input form includes specific questions related to activities of daily living functions, as well as traditional user input fields such as name and date of birth. In this embodiment, there are four areas of specific function: personal care, mobility, instrumental activities, and participation. Recognizing that questions can vary, there are a total of 138 unique questions that can be adjusted based on the activities of daily living, community engagement, social, and educational environments being evaluated within each of the subdomains.

The system gathers information from the user in a unique way by prompting with phrases such as “Could you do the following? (see 301), “Did you do the following”, “Were you responsible for the following?”, and similar questions. Each section of the pre-screening input evaluation is specific to the ADL function being evaluated and is designed to assess pre-injury, post-injury, current, and observed function. This unique questionnaire style improves the determination of capabilities and eliminates uncertainties regarding whether the individual performed certain activities prior to injury or illness. It enhances scoring accuracy and provides a more precise assessment of the individual. It also allows professionals to evaluate from a clinical perspective whether the individual is “choosing not to perform,” thus distinguishing actual capability from behavioral choice, which affects resource requirements for aids, services, and equipment.

The professional, such as a therapist, makes an assessment based on the user's input and records the assessment for further evaluation and analysis (see 302). The professional's assessment, including comments and related data, is saved in the user's file. In one embodiment, the professional has similar prompting phrases during the assessment, such as, “Can you do the following”, “Do you do the following”, “Are you currently responsible for the following?”, and similar questions. “On the administrative side, an admin user has access to all cases based on previously submitted evaluation forms, including access to all case history, data, referral information, and any other available information. To ensure data integrity, only authorized users can modify report details, maintaining the integrity of the assessments.

The system uses national and international statistics to establish a baseline of normal functioning individuals. This baseline can be established for activities of daily living, community engagement, social, and educational environments. By incorporating this data, the system provides an additional clinical and objective layer of specific functional information used to define the baseline. Various government documentation is available to provide baseline statistics, such as from the U.S. Bureau of Labor Statistics, Australian Bureau of Statistics, or similar agencies.

An aspect of the invention is the system's ability to evaluate individual performance in comparison to national datasets or outcomes, as well as published research specific to diagnoses, conditions, or disabilities. These datasets are stored and used for comparative and statistical analysis against data entered about the tested individual. In some embodiments, national datasets are fetched based on an Application Programming Interface (API) or internal data management systems (DMS).

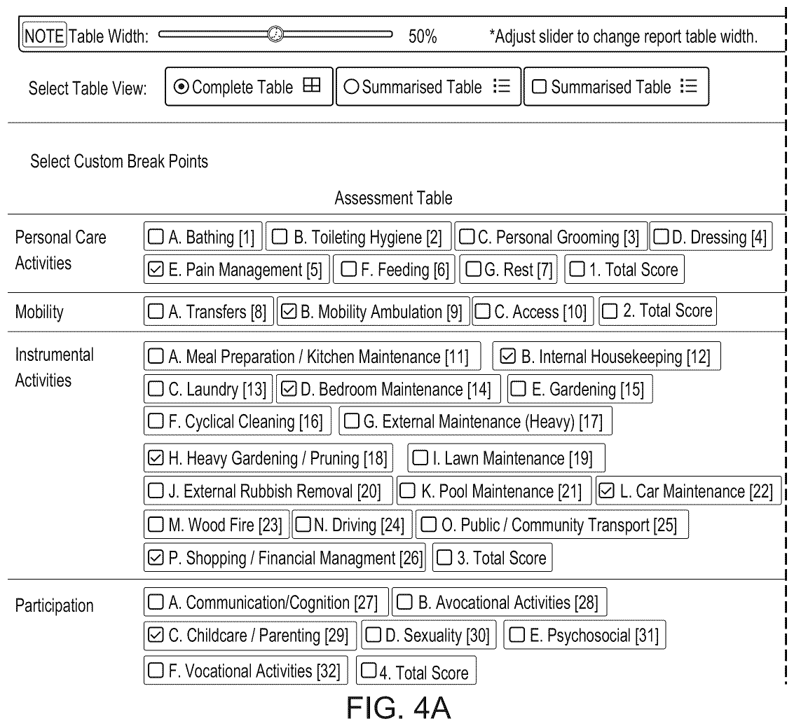

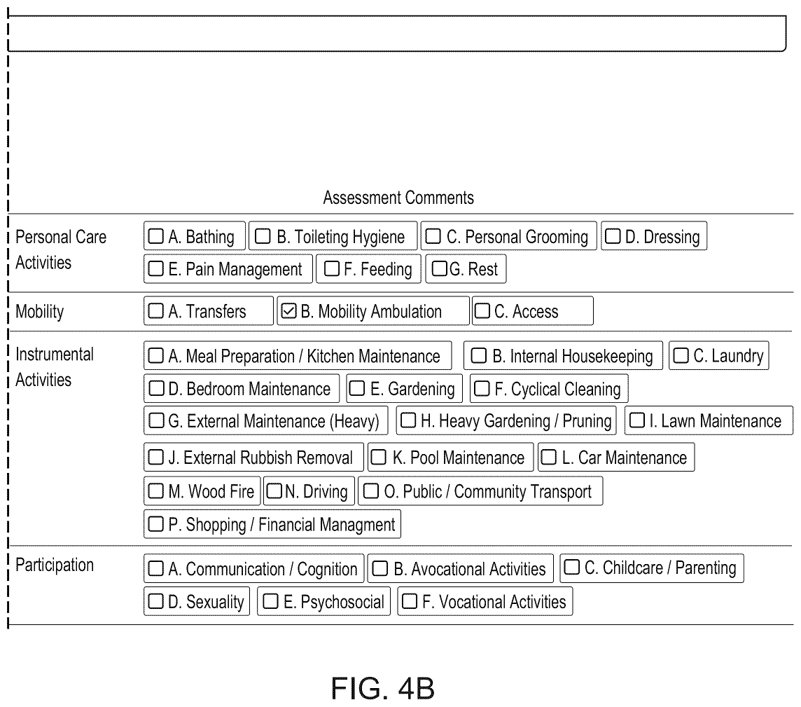

The system is configured for report printing and customization, allowing the administrative user to select sections and set page breaks to generate a professional report easily. This system enables the user to choose which components to include in the report for each individual. An exemplary screenshot is shown in FIGS. 4A-B, displaying radial dials of possible assessments and components to be included in the report.

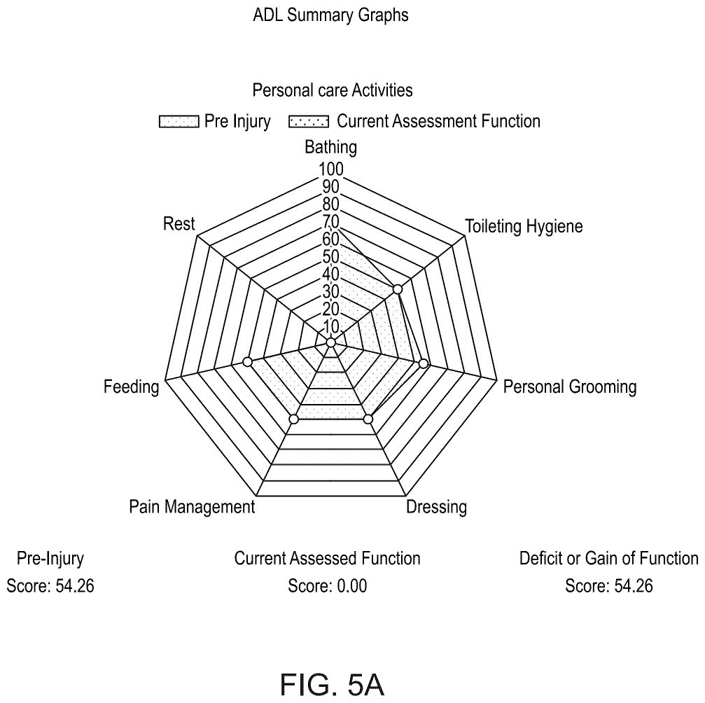

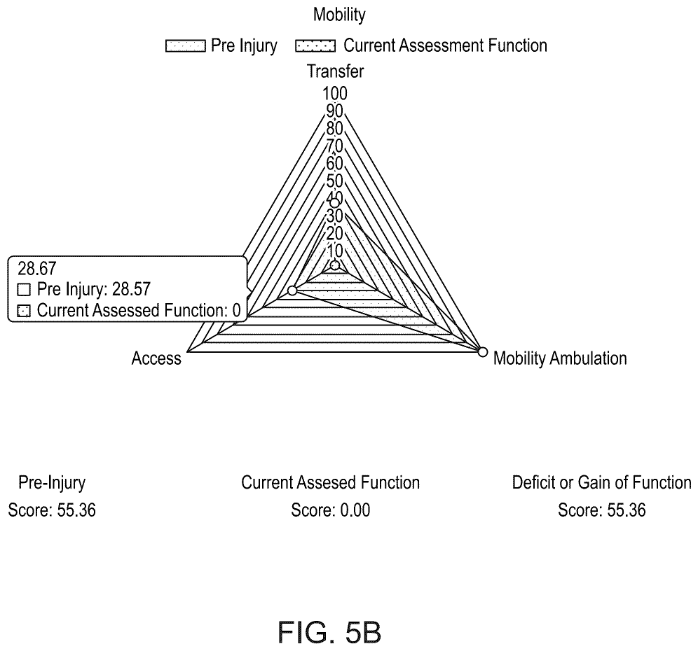

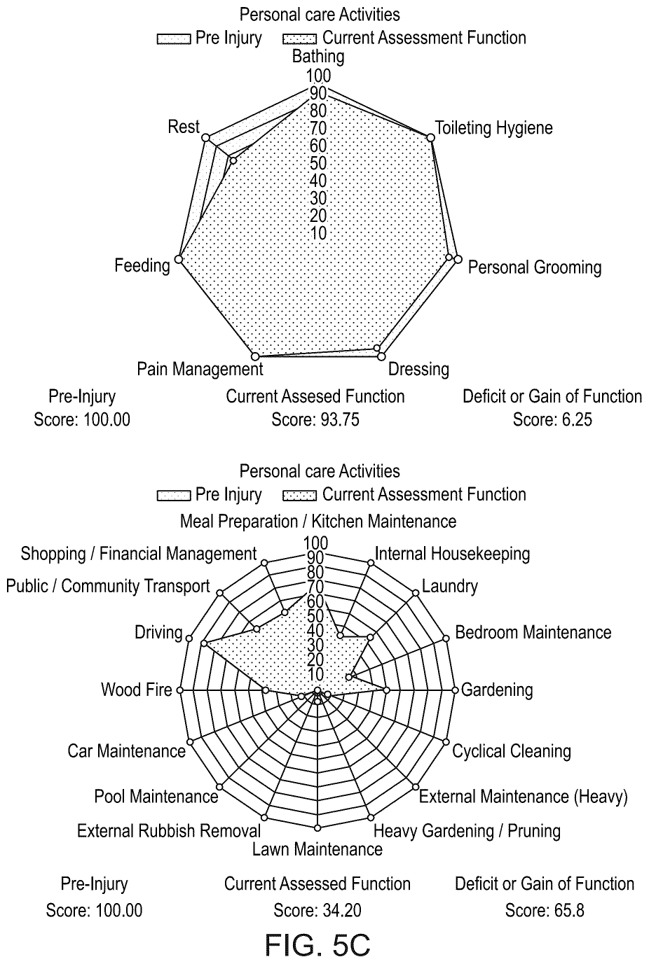

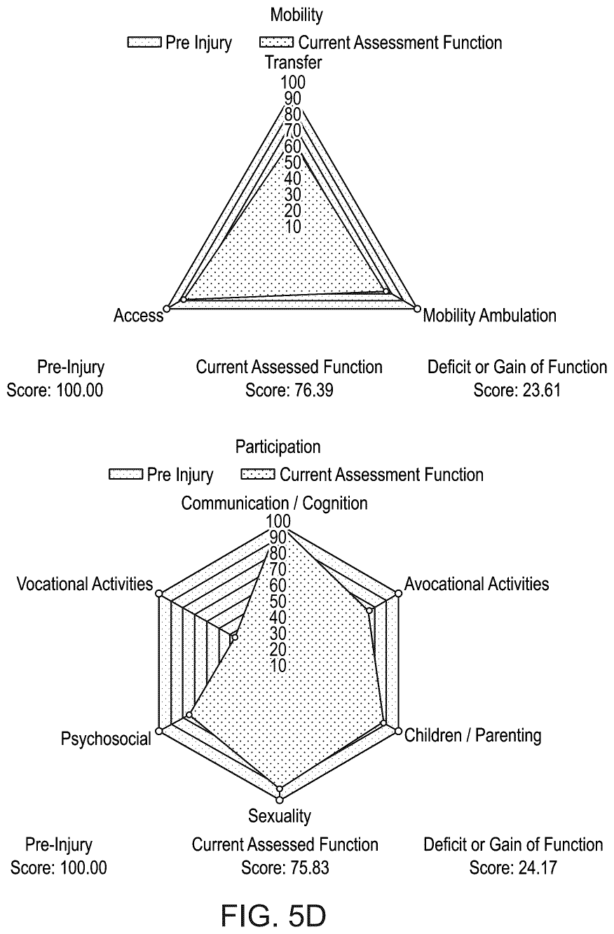

A particularly useful feature of the system is the representation of data, which allows the end user to visually see the results of the pre-injury or disease assessment compared to the objective findings of the trained professional. Unlike other testing systems that only provide scores in a table format, the present system allows users to visualize each of the domains and calculates the specific loss or functional improvements within those domains. Exemplary domain category scores and summary graphs are illustrated in FIGS. 5A-D. The system includes an image capture function, enabling graphical images to be added to individual user reports if the user chooses not to use the system-generated report.

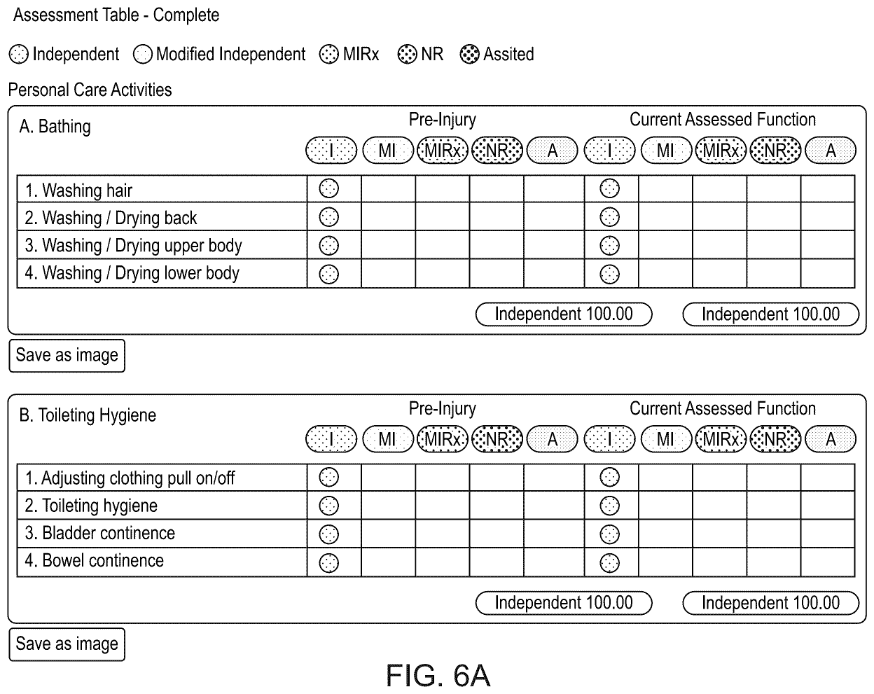

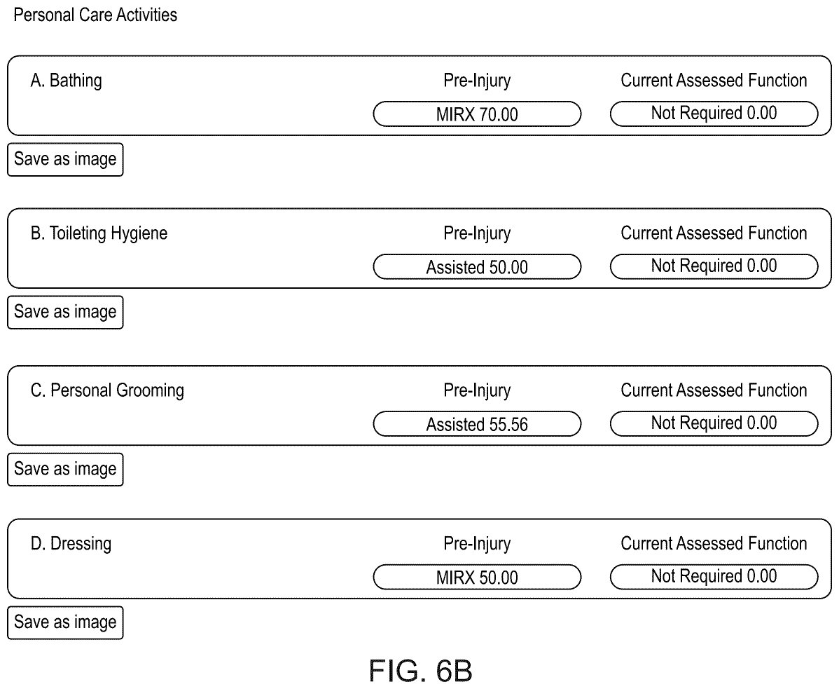

As previously mentioned, there are 138 questionnaires correlated to 138 subdomain components of activities of daily living. Each domain has a specific section and scoring mechanism. Examples of these summaries are shown in FIGS. 6A-B. Referring to FIG. 6B, various domains are listed with their summary scores.

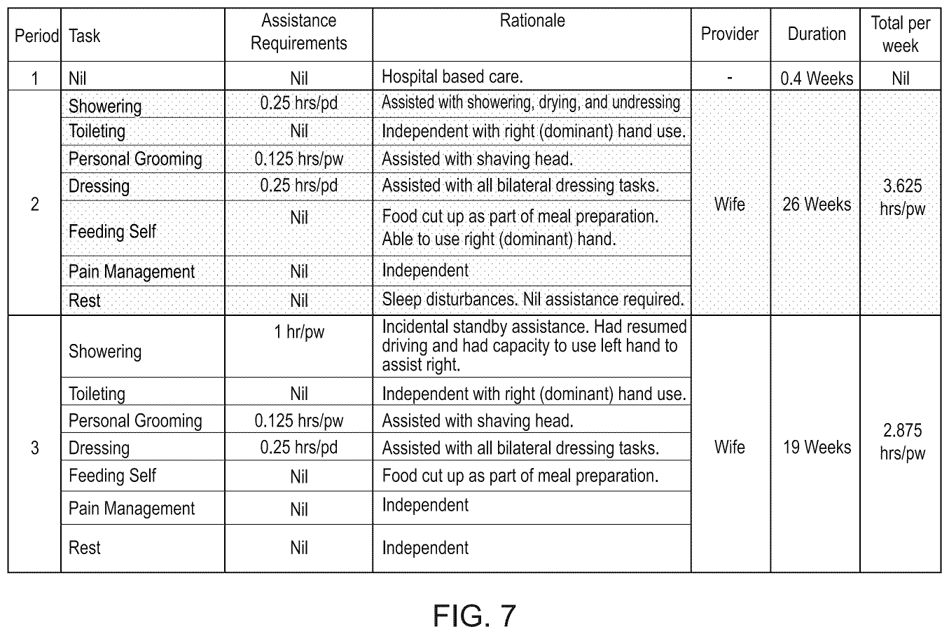

FIG. 7 illustrates a sample table of reviewed subdomains. Based on schedules, standards, or normative data, the schedule of resources and rationale is calculated into the report. Resources are calculated based on current capabilities compared to those expected as a result of functional gain or loss from the baseline assessment.

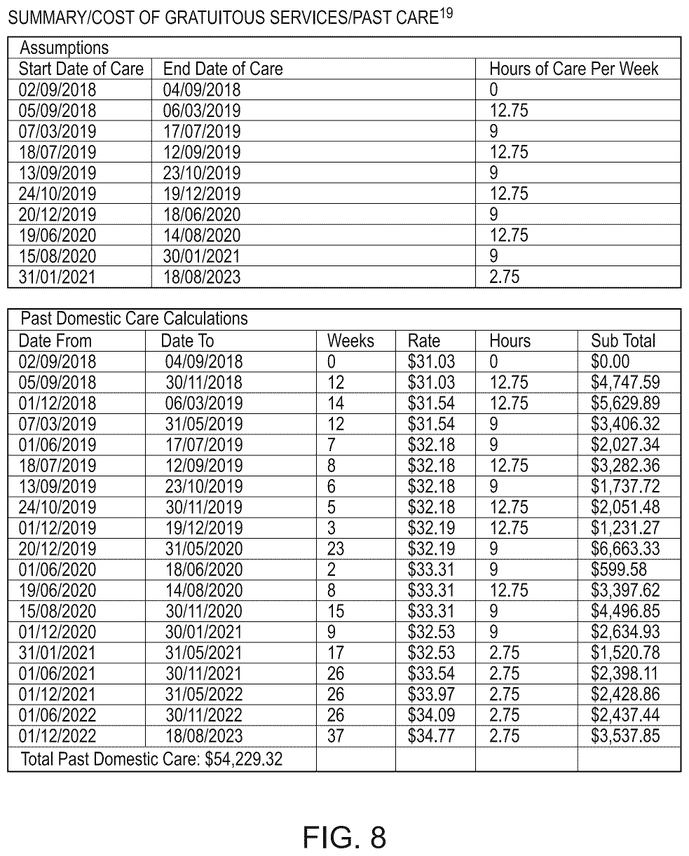

FIG. 8 illustrates a sample table of cost analysis for services, resources, aids, and equipment calculations. Based on schedules, standards, or normative data, the cost analysis incorporates the latest fee schedules or allowances for services, resources, aids, and equipment. These costs are calculated based on current capabilities compared to those expected from the baseline assessment.

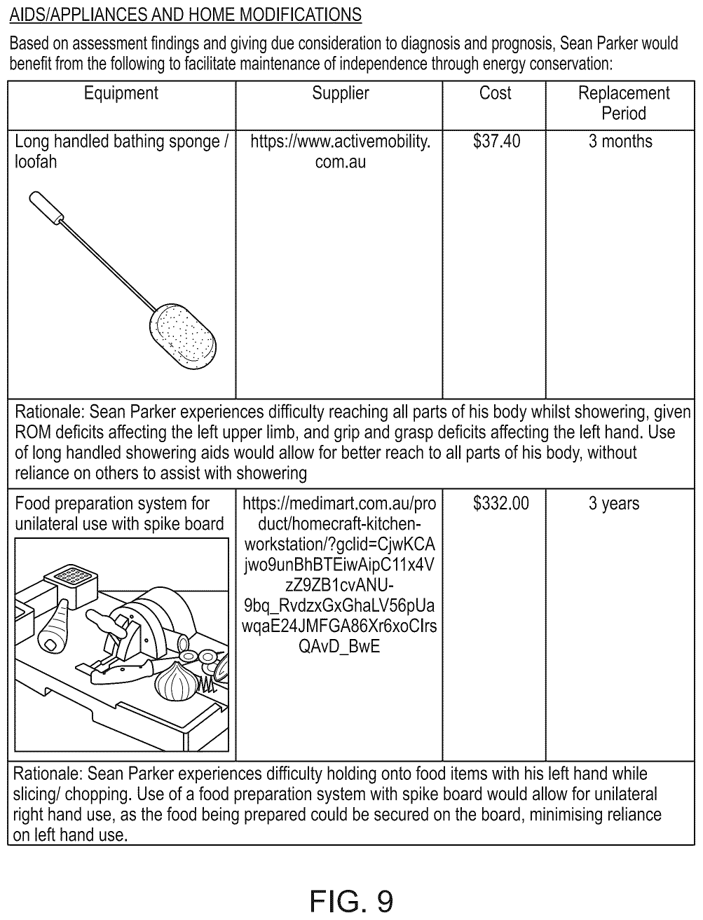

FIG. 9 illustrates a sample table of aids and home modifications. Based on the needs or requirements of the individual, the professionally trained evaluator has access to a catalog and listing of equipment, suppliers, and costs that can be added to the report. This feature is significant as a built-in data source. As new sources are added, the database is updated to keep current with the market and includes equipment found by multiple providers.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. In other words, the terminology and phraseology used in this description and the abstract are for illustrative purposes and should not be considered limiting. Therefore, while exemplary illustrative embodiments of the invention have been described, numerous variations and alternative embodiments will occur to those skilled in the art. Such variations and alternative embodiments are contemplated, and can be made without departing from the spirit and scope of the invention.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

What is claimed is:

1. A software-as-a-service (SaaS) system for determining specific calculated individual functional changes in the level of resources needed using unique identifiers within activities of daily living, community engagement, social participation, and educational environments, the system comprising:
an input interface configured to receive data collected from an individual user regarding previous capabilities identified by unique identifiers within specific functional domains including activities of daily living, community engagement, social participation, and educational activities;
wherein the input interface is configured to receive observed capability data provided by a trained professional corresponding to the unique identifiers;
a first database configured to store normative data comprising research-based datasets of functional levels corresponding to the unique identifiers within the specific functional domains;
a second database configured to store fee schedules, costs, aids, home modifications, resource time requirements of normal populations, and diagnosis-specific physical change expectations;
a processor configured to:
(a) associate the received individual data and professional data with the unique identifiers to map functional capabilities accurately;
(b) compare the individual data and the observed capability data to the normative data for each unique identifier to determine deviations from normative functional levels;
(c) calculate specific percentage losses or gains in function for the individual user for each unique identifier based on the comparison;
(d) determine the level of resource need by integrating the calculated functional changes with time components, resource requirements, and equipment needs corresponding to each unique identifier;
(e) generate an outcome report that includes the calculated percentage losses or gains, graphical representations of changes in function, and recommendations for resources, equipment, services, and treatments;
(f) project long-term changes in status, function, and resource requirements based on diagnosis-specific expectations;
an output interface configured to output the outcome report to authorized users.

2. The system of claim 1, wherein the unique identifiers comprise specific tasks or activities that uniquely identify functional capabilities and corresponding resource needs within the functional domains.

3. The system of claim 1, wherein the input interface presents questionnaires to the individual user formatted with prompts including, “Could you do the following?,” “Did you do the following?,” and “Were you responsible for the following?” to assess pre-injury or baseline capabilities accurately.

4. The system of claim 1, wherein the processor combines the data collected from the individual user and the observed capability data provided by the trained professional to assess changes in functional levels more accurately.

5. The system of claim 4, wherein the processor is configured to use the data collected from the individual user and the observed capability data and apply a scoring process to objectively assess and score the current status of the individual, integrate the score with the normative data and diagnosis data to perform priority data calculations for determining resource needs.

6. The system of claim 1, wherein the outcome report includes graphical representations to visually explain changes in function based on specific domains and subdomains.

7. The system of claim 1, wherein the processor calculates required resources, services, equipment, and associated costs by integrating pre- and post-function time requirements and accessing current fee schedules from the database.

8. The system of claim 1, wherein the projection of long-term changes utilizes diagnosis-specific normative data to anticipate future functional changes and resource requirements.

9. The system of claim 1, wherein the system is utilized for individual user baseline testing, periodic reassessments, evaluations by government agencies to determine functional changes over time, and determination of resource requirements and disability payouts in medical legal cases.

10. A method for determining specific calculated individual functional changes in the level of resource need using unique identifiers within activities of daily living, community engagement, social, and educational environments, the method comprising:
receiving, via an input interface of a software-as-a-service (SaaS) system, data collected from an individual user regarding previous capabilities identified by unique identifiers within specific functional domains including activities of daily living, community engagement, social participation, and educational activities;
receiving, via the input interface, observed capability data corresponding to the unique identifiers, the capability data provided by a trained professional;
associating the received individual user data and the professional data with the unique identifiers to accurately map functional capabilities;
accessing normative data from a first database, the normative data comprising research-based datasets of functional levels corresponding to the unique identifiers within the specific functional domains;
accessing additional data from a second database, the additional data comprising fee schedules, costs, aids, home modifications, resource time requirements of normal populations, and diagnosis-specific physical change expectations;
comparing the individual user data and the observed capability data to the normative data for each unique identifier to determine deviations from normative functional levels;
calculating specific percentage losses or gains in function for the individual user for each unique identifier based on the comparison;
determining the level of resource need by integrating the calculated functional changes with time components, resource requirements, and equipment needs corresponding to each unique identifier;
generating an outcome report, and
outputting the outcome report via an output interface.

11. The method of claim 10, wherein the outcome report includes the calculated percentage losses or gains; graphical representations to visually explain changes in function based on specific domains and subdomains; and recommendations for resources, equipment, services, and treatments.

12. The method of claim 10, further providing the step of projecting long-term changes in status, function, and resource requirements based on diagnosis-specific expectations.

13. The method of claim 10, wherein the unique identifiers comprise specific tasks or activities that uniquely identify functional capabilities and corresponding resource needs within the functional domains.

14. The method of claim 10, wherein the step of receiving data from the individual user includes presenting questionnaires formatted with prompts including, Could you do the following?,” “Did you do the following?,” and “Were you responsible for the following?” to accurately assess pre-injury or baseline capabilities.

15. The method of claim 10, wherein associating the data includes combining the data collected from the individual user and the observed capability data provided by the trained professional to more accurately assess changes in functional levels.

16. The method of claim 14, further comprising a scoring process applied to the individual user data and observed capability data to objectively assess the current status of the individual; the scores are integrated with the normative data and diagnosis data to provide a comprehensive dataset used by the processor to perform priority data calculations for determining resource needs.

17. The method of claim 10, wherein generating the outcome report includes creating graphical representations to visually explain changes in function based on specific domains and subdomains.

18. The method of claim 10, wherein determining the level of resource need includes calculating required resources, services, equipment, and associated costs by integrating pre- and post-function time requirements and accessing current fee schedules from the second database.

19. The method of claim 13, wherein projecting long-term changes utilizes diagnosis-specific normative data to anticipate future functional changes and resource requirements.

20. The method of claim 10, wherein the method is utilized for individual user baseline testing, periodic reassessments, evaluations by government agencies to determine functional changes over time, and determination of resource requirements and disability payouts in medical legal cases.
